(19) United States
(12) Patent Application Publication
(10) Pub. No.: US 2026/0014080 A1
(43) Pub. Date: Jan. 15, 2026

(54) LIPOSOMAL PREPARATION WITH ENCAPSULATED HORMONES, METHOD OF PRODUCTION AND USE THEREOF

(71) Applicants: Gina VAN BOGAERT, Antwerpen (BE); PURNA PHARMACEUTICALS NV, Puurs (BE)

(72) Inventors: Gina VAN BOGAERT, Antwerpen (BE); Bart PEETERS, Puurs (BE)

(21) Appl. No.: 18/866,977

(22) PCT Filed: May 17, 2023
(86) PCT No.: PCT/IB2023/055076

§ 371 Date: Nov. 18, 2024

(30) Foreign Application Priority Data
May 18, 2022 (BE) 2022/5374

(51) Int. Cl. A61K 9/1277; A61K 31/568; A61K 31/573; A61K 47/10; A61K 47/32

(52) U.S. Cl. CPC A61K 9/1277; A61K 31/568; A61K 31/573; A61K 47/10; A61K 47/32

(57) ABSTRACT

The present invention relates to a method for producing a liposomal preparation by emulsification, the preparation containing liposomes with encapsulated hormones, comprising: a first emulsification step of emulsifying a first composition, the first composition comprising water, propylene glycol, an emulsifier and a hormone to be encapsulated; characterized in that for the emulsification of the first emulsification step, a rotor-stator emulsifier is used, which is provided with a stator with perforations with a diameter between 0.8 and 5 mm, and wherein the rotational speed is at least 6000 rpm. The invention also relates to a liposomal preparation comprising liposomes with encapsulated hormones or esters thereof, and a liposomal preparation for use in hormone therapy or for use in the treatment of hypogonadism and/or adrenal insufficiency.

### TECHNICAL FIELD

The invention relates to a method for producing a liposomal preparation, to a liposomal preparation and the use of such preparation.

### PRIOR ART

Liposomes are spherical vesicles with at least one lipid bilayer or double layer. Liposomes can be used as vehicles for the delivery of nutrients and pharmaceutical drugs, such as lipid nanoparticles in mRNA vaccines and DNA vaccines. Liposomes can be prepared by disrupting biological membranes, for example by sonication.

Liposomes are mostly composed of phospholipids, especially phosphatidylcholine, but can also contain other lipids, such as egg phosphatidylethanolamine, as long as they are compatible with the structure of the lipid bilayer.

The main types of liposomes are the multilamellar vesicle (multilamellar liposome vesicle: MLV, with multiple lipid bilayers in lamellar phase), the small unilamellar liposome vesicle (SUV, with one lipid bilayer), the large unilamellar vesicle (large unilamellar liposome vesicle: LUV), and the cochleate vesicle. A less desirable form are multivesicular liposomes, where one vesicle contains one or more smaller vesicles.

Liposomes are used, among other things, for liposomally encapsulated molecules applied topically or directly to the skin for transdermal delivery of the same molecules with a systematic effect in mind. Their lipid bilayer structure resembles that of natural cell membranes and allows them to influence the fluidity of cell membranes, to fuse with them and to migrate between cells and cell membranes.

Multilamellar liposomes can deliver drugs through the dermis, epidermis and stratum corneum layers of the skin within half an hour in significantly higher amounts than conventional preparations without liposomes.

WO2010096886 already describes a method for preparing liposomes with encapsulated hydrocortisone, a composition of a liposomal gel and its use for transdermal administration. In this process, liposomes with encapsulated hydrocortisone are emulsified or suspended in a modified gel. To this end, the liposomes are mixed in a suitable mixer together with gel matrices that form a polymeric three-dimensional structure around the liposomes, such as, for example, a hydrogel. EP3025732 also describes a method of preparing a liposomal preparation for administering encapsulated drugs by emulsification. Further methods for producing liposomal preparations are also known from e.g. WO0120990. In addition, liposomal preparations are discussed in US2020038325, CA2366998 and EP2308468.

A problem with the known liposomal preparations with drugs encapsulated in the liposomes is that serum levels of the drug in question in the blood do not remain relatively constant at a desired level over a prolonged period of time when applied to a patient. As a result, the preparation must be re-administered very regularly, several times a day, regardless of whether this administration is carried out transdermally, per os or intravenously. Because these liposomes need to be administered multiple times, it is difficult for many patients to remain treatment compliant, i.e. the willing and continued following of the treatment prescribed by a doctor by a patient. Reduced treatment compliance can lead to an increased risk of disease, mortality and higher costs.

There is a need for an improved preparation suitable for drug administration, whereby serum levels of the drug in the blood remain relatively stable for a longer period of time, so that fewer applications are required.

The present invention aims to find a solution for at least some of the above problems.

### SUMMARY OF THE INVENTION

The invention relates to a method for producing a liposomal preparation by emulsification, the preparation containing liposomes with encapsulated hormones or ester thereof according to claim 1. The method comprises the use of a rotor-stator emulsifier provided with a stator with perforations with specific characteristics.

In a second aspect, the invention relates to a liposomal preparation comprising liposomes with encapsulated hormones or esters thereof according to claim 10.

In a third aspect, the invention relates to a liposomal preparation for use in hormone therapy or for use in the treatment of a patient requiring hormone therapy according to claim 14.

In a fourth aspect, the invention relates to a liposomal preparation for use in the treatment of hypogonadism and/or adrenal insufficiency according to claim 15.

Preferred embodiments are discussed in the dependent claims.

The advantage of the method for producing a liposomal preparation, the liposomal preparation and the use of the liposomal preparation according to the present invention is that such preparation releases the encapsulated hormones or esters thereof particularly efficiently. In particular, a prolonged release is possible, preferably from at least 8 to 32 hours. As a result, the number of applications can be reduced, which promotes patient compliance and saves costs.

### DESCRIPTION OF THE FIGURES

FIG. 1 shows schematically and in front view a rotor-stator emulsifier used in the method for producing a liposomal preparation according to an embodiment of the invention.

FIG. 2 shows schematically and in side view a rotor-stator emulsifier used in the method for producing a liposomal preparation according to an embodiment of the invention.

FIG. 3 shows an experimental stator with 1.6 mm perforations as used in the method for producing a liposomal preparation according to an embodiment of the invention and as discussed in the examples below.

FIG. 4 shows a conventional stator with 9 mm perforations, as discussed in the examples below.

FIG. 5 shows a standard calibration curve for determining unknown testosterone concentrations in the samples from the IVPT tests.

FIG. 6 shows an HPLC result of a tested sample (50 μg/ml testosterone+a skin extract solution) by the developed validation method at a flow rate of 0.5 ml/min.

### DETAILED DESCRIPTION

The invention relates to a method for producing a liposomal preparation by emulsification, the preparation containing liposomes with encapsulated hormones or ester thereof. The method comprises the use of a rotor-stator emulsifier provided with a stator with perforations with specific characteristics. The invention also relates to a liposomal preparation and the use of a liposomal preparation. The main advantage of this is that such a preparation releases the encapsulated hormones or esters thereof particularly efficiently. In particular, a prolonged release is possible, preferably lasting at least 8 to 32 hours. As a result, the number of applications per day, week or month can be reduced, which promotes patient compliance and saves costs.

### Definitions

Unless otherwise defined, all terms used in the description of the invention, including technical and scientific terms, have the meanings commonly understood by those skilled in the art of the invention. For a better understanding of the description of the invention, the following terms are explained explicitly.

In this document, “a” and “the” refer to both the singular and the plural, unless the context presupposes otherwise. For example, “a segment” means one or more segments.

When the term “around” or “about” is used in this document with a measurable quantity, a parameter, a duration or moment, and the like, then variations are meant of approx. 20% or less, preferably approx. 10% or less, more preferably approx. 5% or less, even more preferably approx. 1% or less, and even more preferably approx. 0.1% or less than and of the quoted value, insofar as such variations are applicable in the described invention. However, it must be understood that the value of a quantity used where the term “about” or “around” is used, is itself specifically disclosed.

The terms “comprise,” “comprising,” “consist of,” “consisting of,” “provided with,” “have,” “having,” “include,” “including,” “contain,” “containing” are synonyms and are inclusive or open terms that indicate the presence of what follows, and which do not exclude or prevent the presence of other components, characteristics, elements, members, steps, as known from or disclosed in the prior art.

Quoting numeric intervals by the endpoints includes all integers, fractions, and/or real numbers between the endpoints, including those endpoints.

“Hormone therapy” or “hormonal therapy”, in the current context, refers to any type of therapy that uses hormones in medical treatment. Treatment with hormone antagonists may also be referred to as hormonal therapy or anti-hormone therapy. The most common classes of hormone therapy are oncological hormone therapy, hormone replacement therapy (for menopause), androgen replacement therapy (ART, including testosterone replacement therapy (TRT)), oral contraceptive pills, and transgender hormone therapy.

Different types of hormone therapy are discussed below and may all form part of the object of the current application:

“Hormone replacement therapy” (HRT), also known as menopausal hormone therapy (MHT), is intended for people suffering from menopausal symptoms. It is based on the idea that the treatment can prevent discomfort caused by reduced circulating estrogen and progesterone hormones, or in the case of surgically or prematurely menopausal individuals, that it can prolong life and reduce the incidence of dementia. It involves taking one or more of a group of medications designed to artificially increase hormone levels. The main types of hormones are estrogen, progesterone or progestogens, and sometimes testosterone. It is often referred to as “treatment” rather than therapy. “Hormone replacement therapy” for people with hypogonadism and intersex conditions (e.g. Klinefelter syndrome, Turner syndrome).

“Androgen Replacement Therapy” (ART) in men with low testosterone levels due to disease or aging. It is a hormone treatment often prescribed to counteract the effects of male hypogonadism or for men who have lost their testicular function due to disease, cancer or other causes. It is sometimes used for late-onset hypogonadism (the so-called “andropause”).

“Transgender hormone therapy” for transgender people introduces sex steroids associated with the sex the patient identifies with (specifically testosterone for transgender men and estrogen for transgender women). Some intersex and non-binary individuals may also undergo hormone therapy. Cross-sex hormone treatments for transsexuals are divided into two main types: feminizing hormone treatments and masculinizing hormone treatments. Feminizing hormone therapy in gender reassignment therapy for transgender women, and Masculinizing hormone therapy in gender reassignment therapy for transsexual men.

“Hormonal therapy for cancer,” such as androgen deprivation therapy for men with prostate cancer, estrogen deprivation therapy for women with estrogen receptor-positive breast cancer, and high-dose estrogen therapy for women with estrogen receptor-positive breast cancer.

“Chemical castration” of men or sex offenders with paraphilias or hypersexuality.

“Growth hormone therapy” for a growth hormone deficiency.

“Thyroid hormone replacement” in hypothyroidism.

“Antithyroid therapy” in hyperthyroidism.

“Replacement of glucocorticoids and/or mineralocorticoids” in conditions such as Addison's disease.

“Antiglucocorticoid therapy” in Cushing's syndrome.

“Insulin therapy” in type 1 diabetes.

“Oral contraceptive pills” for various purposes, including birth control.

The term “patient” or “subject” refers to animals, preferably warm-blooded animals, more preferably vertebrates, even more preferably mammals, even more preferably primates, and especially human patients and non-human mammals and primates. Preference is given to human patients. The term includes subjects in need of treatment, more specifically subjects who would benefit from treatment for a particular condition. Such subjects may comprise, without limitation, those diagnosed with said condition, those at risk of developing said condition, and/or those in whom said condition is to be prevented.

The term “treatment” refers both to therapeutic treatment and to prophylactic or preventative measures, the purpose of which is to prevent or delay (reduce) the targeted pathological condition or disorder. Those in need of treatment include those who already have the condition, as well as those at risk of developing the condition or in whom the condition needs to be prevented. The terms “treat” or “treatment” as used herein include alleviating, reducing or ameliorating the symptoms of the disease or condition, preventing additional symptoms, ameliorating or preventing the underlying metabolic causes of the symptoms, inhibition of the disease or condition, e.g. stopping the progression of the disease or condition, alleviating the disease or condition, causing regression of the disease or condition, alleviating a condition caused by the disease or condition, or stopping the symptoms of the disease or condition.

“Organic solvents” are carbonaceous substances capable of dissolving or dispersing one or more other substances. Many classes of chemicals are used as organic solvents, including aliphatic hydrocarbons, aromatic hydrocarbons, amines, esters, ethers, ketones, and nitrated or chlorinated hydrocarbons. Organic solvents are used in many industries. They are used in paints, varnishes, lacquers, adhesives, glues, degreasing and cleaning agents, and in the production of dyes, polymers, plastics, textiles, printing inks, agricultural products and pharmaceuticals.

### DESCRIPTION

In a first aspect, the invention relates to a method for producing a liposomal preparation by emulsification, wherein the preparation contains liposomes with encapsulated hormones or ester thereof. The method comprises at least a first emulsification step in which a first composition is emulsified. This first composition comprises water, propylene glycol, an emulsifier and a hormone to be encapsulated or an ester of such hormone. For the emulsification of this first emulsification step, a rotor-stator emulsifier is used, which is provided with a stator with perforations with a diameter between 0.8 and 5 mm. The rotational speed, i.e. the speed of rotation of the rotor inside the stator, should be at least 6000 rpm.

An example of such a rotor-stator emulsifier is a Silverson® type L5M-A. It is equipped with a rotating impeller as a rotor, surrounded by an annular stator, also called a shear head, with perforations, where the rotating impeller ensures that the mixture to be emulsified is sucked in under the rotor, and then centrifugally pressing the mixture out through the perforations of the stator, creating high shear forces or Van Der Waals forces.

The advantage of the method of the present invention is that the resulting liposomal preparation when administered to a patient, such as by transdermal, intravenous or per os administration, provides a stable, slow and sustained release of the encapsulated hormone. In order to obtain such a preparation with liposomes and prolonged release, the diameter of the perforations of the stator is essential. It should be between 0.8 and 5 mm, preferably between 0.8 and 3 mm. Most preferably, the diameter of the perforations is about 1.6 mm. The rotational speed of at least 6000 rpm optimizes the resulting liposomal preparation.

This stable, slow and prolonged release of the encapsulated hormone means that constant serum levels of the hormone in a patient's blood are achieved, and that once they are brought to a desired level they remain relatively constant for at least 8 hrs, at least 10 hrs, at least 12 hrs, at least 14 hrs, at least 16 hrs, at least 18 hrs, at least 20 hrs, at least 22 hrs, at least 24 hrs, at least 26 hrs, at least 28 hrs, at least 30 hrs, or at least 32 hrs, preferably for at least 8 to 32 hrs. As a result, a single application per day may be sufficient to keep serum levels of the hormone constant, or sufficiently constant. Reducing the number of necessary applications per day promotes patient compliance and saves costs.

The distance between the perforations in the stator is preferably such that the smallest distance from a first perforation to a second perforation is 0.5 to 1.5 mm, preferably about 1.0 mm. This smallest distance can be measured as the smallest distance from an edge of a first perforation to an edge of a second perforation.

The encapsulated hormone can be any type of hormone, or any type of ester thereof, which can be encapsulated in a liposome of the liposomal preparation of the present invention. According to an embodiment, the hormone is a sex hormone, preferably selected from the group of testosterone, hydrocortisone, estradiol and progesterone, or ester thereof.

Testosterone is an androgen with active metabolites dihydrotestosterone (DHT) and estradiol (E2). Oestradiol or estradiol (17β-estradiol) falls into the group of estrogens. Hydrocortisone is the name for the hormone cortisol when supplied as a medicine. Cortisol is a steroid hormone, in the glucocorticoid class of hormones. Progesterone (P4) is an endogenous steroid and progestogen sex hormone.

According to an embodiment, the esters of hormones that are encapsulated are esters of steroids or steroid esters.

According to an embodiment, the esters of hormones that are encapsulated are esters of androgens or androgen esters. These can be esters of natural or synthetic androgens. According to an embodiment, the esters of hormones that are encapsulated are testosterone esters. Testosterone esters comprise testosterone caproate, testosterone cypionate, testosterone decanoate, testosterone enanthate, testosterone isobutyrate, testosterone isocaproate, testosterone phenylpropionate, testosterone propionate, testosterone undecanoate, testosterone acetate, testosterone cyclohexylpropionate, testosterone enanthate benzilic acid hydrazone, testosterone hexahydrobenzoate, testosterone hexahydrobenzyl carbonate, testosterone hexyloxy phenylpropionate, testosterone ketolaurate, testosterone nicotinate, testosterone phenylacetate, testosterone phosphate, testosterone undecylenate, testosterone valerate, testosterone buciclate, polytestosterone phloretin phosphate, testosterone 17β-(1-((5-(aminosulfonyl)-2-pyridinyl)carbonyl)-L-proline) (EC586), testosterone acetate butyrate, testosterone acetate propionate, testosterone benzoate, testosterone butyrate, testosterone diacetate, testosterone dipropionate, testosterone formate, testosterone isovalerate, testosterone palmitate, testosterone phenylbutyrate, testosterone stearate, testosterone succinate and testosterone sulfate. Dihydrotestosterone esters comprise androstanolone benzoate, androstanolone enantate, androstanolone propionate, androstanolone valerate, dihydrotestosterone acetate, dihydrotestosterone butyrate, dihydrotestosterone formate, and dihydrotestosterone undecanoate.

Preferably, the encapsulated hormone is testosterone propionate.

According to an embodiment, the esters of encapsulated hormones are esters of estrogens, for example, estradiol esters, estrone esters, estriole esters, ethinylestradiol esters, and esters of other estrogenic steroids.

Examples of estradiol esters are estradiol acetate, estradiol benzoate, estradiol cypionate, estradiol dipropionate, estradiol enanthate, estradiol undecylate, estradiol valerate, polyestradiol phosphate, cloxestradiol acetate, estradiol benzoate butyrate, estradiol butylacetate, estradiol dibutyrate, estradiol dienannate, estradiol di-undecylate, estradiol diundecylenate, estradiol furoate, estradiol hemisuccinate, estradiol hexahydrobenzoate, estradiol palmitate, estradiol phenylpropionate, estradiol pivalate, estradiol propionate (estradiol 17β-propionate), estradiol propoxyphenylpropionate, estradiol stearate, estradiol sulfate, estramustine phosphate (estradiol 3-normustine 17β-phosphate), estradiol 3-furoate, estradiol 3-propionate, estradiol 17β-(1-(4-(aminosulfonyl)benzoyl)-L-proline), estradiol acetate benzoate, estradiol 17β-acetate, estradiol 17β-benzoate, estradiol acetylsalicylate (estradiol 3-acetylsalicylate), estradiol anthranilate (estradiol 3-anthranilate), estradiol arachidonate, estradiol benzoate cyclooctenyl ether (estradiol 3-benzoate 17β-cyclooctenyl ether; EBCO), estradiol caprylate (estradiol octanoate), estradiol cyclooctyl acetate (E2CoA), estradiol decanoate (estradiol 17β-decanoate), estradiol diacetate, estradiol dibenzoate, estradiol dicypionate, estradiol dioleate, estradiol dipalmitate, estradiol distearate, estradiol disulfate, estradiol glucuronide, estradiol sulfate glucuronide, estradiol linoleate, estradiol oleate, estradiol phosphate, estradiol salicylate (estradiol 3-salicylate), estradiol sulfamate (estradiol-3-O-sulfamate), estradiol undecylenate, estrapronikate (estradiol 3-propionate 17β-nicotinate), orestrate (estradiol 3-propionate 17β-(1-cyclohexenyl) ether), alestramustine (estradiol 3-(bis(2-chloroethyl)carbamate), 17-ester with L-alanine), estradiol mustard (chlorphenacyl estradiol diester), and estromustine (estrone 17β-3-N-bis(2-chloroethyl)carbamate, estrone-cytostatic complex).

Examples of estrone esters are estrone acetate, estrone sulfate, estropipate, estrone tetraacetylglucoside, estrone benzoate, estrone cyanate, estrone enanthate, estrone benzilic acid hydrazone, estrone glucuronide, estrone phosphate, estrone propionate, estrone sulfamate (estrone-3-O-sulfamate), and estrone oleate.

Examples of estriol esters are estriol 3-glucuronide, estriol acetate benzoate, estriol glucuronide, estriol succinate, estriol sodium succinate, estriol sulfate, estriol sulfate glucuronide, estriol tripropionate, polyestriol phosphate, estriol dihexanoate, estriol dipropionate, estriol phosphate (E3P), estriol sulfamate (estriol-3-O-sulfamate), and estriol triacetate.

Examples of ethinylestradiol esters are ethinylestradiol sulfonate (ethinylestradiol 3-isopropyl sulfonate), ethinylestradiol benzoate—the 3-benzoate ester of ethinylestradiol, ethinylestradiol N,N-diethylsulfamate—the 3-(N,N-diethyl)sulfamate ester of ethinylestradiol, ethinylestradiol pyrrolidinosulfonate—the 3-pyrrolidinosulfonate ester of ethinylestradiol, ethinylestradiol sulfamate—the 3-sulfamate ester of ethinylestradiol, and ethinylestradiol sulfate.

An example of esters of other steroidal estrogens is hydroxyestrone diacetate.

According to an embodiment, the esters of hormones that are encapsulated are esters of corticosteroids, including natural and synthetic corticosteroids. Natural corticosteroids include, for example, esters of desoxycortone, hydrocortisone esters, and esters of other natural corticosteroids.

Examples of esters of desoxycortone are desoxycortone acetate, desoxycortone cypionate, desoxycortone enanthate, desoxycortone glucoside, and desoxycortone pivalate.

Examples of esters of hydrocortisone are Benzodrocortisone (hydrocortisone 17-benzoate), hydrocortamate (hydrocortisone 21-(diethylamino)acetate), hydrocortisone aceponate (hydrocortisone 21-acetate 17α-propionate), hydrocortisone acetate, hydrocortisone bendazac, hydrocortisone buteprate (hydrocortisone 17α-butyrate 21-propionate) hydrocortisone butyrate (hydrocortisone 17α-butyrate), hydrocortisone 21-butyrate, hydrocortisone cypionate (hydrocortisone cyclopentane propionate), hydrocortisone phosphate, hydrocortisone succinate (hydrocortisone hemisuccinate), hydrocortisone tebutate, hydrocortisone valerate, and hydrocortisone xanthic acid.

Examples of esters of other natural corticosteroids are 11-Dehydrocorticosterone acetate, cortifen (cortodoxone chlorophenacyl ester), cortisone acetate, corticosterone acetate, corticosterone benzoate, and cortodoxone acetate.

Examples of esters of synthetic corticosteroids are esters of beclomethasone, esters of betamethasone, esters of clocortolone, esters of dexamethasone, esters of fluocinolone acetonide, esters of fluocortolone, esters of fluprednisolone, esters of methylprednisolone, esters of prednisolone, esters of prednisone, esters of tixocortol, and esters of triamcinolone acetonide.

According to an embodiment, the esters of encapsulated hormones are esters of progesterone or progesterone derivatives. These comprise esters of 17α-hydroxyprogesterone derivatives including chlormadinone acetate, cyproterone acetate, hydroxyprogesterone caproate, medroxyprogesterone acetate, megestrol acetate, acetomepregenol, anagestone acetate, chlormethenmadinone acetate, flumedroxone acetate, hydroxyprogesterone acetate, hydroxyprogesterone heptanoate, methenmadinone acetate, and pentagestron acetate; esters of 19-norprogesterone derivatives including gestonorone caproate, nomegestrol acetate, and segesterone acetate; and esters of 19-nortestosterone derivatives including etynodiol diacetate, norethisterone acetate, norethisterone enanthate, norgestimate, and quingestanol acetate.

The desired level at which the serum levels of a hormone in a patient's blood are preferably kept constant by the stable, slow and prolonged release of the encapsulated hormone in the liposomal preparation varies per hormone.

Preferably, the desired minimum level of total testosterone in the blood of an adult male is 800 ng/dL and the desired minimum level of free testosterone in the blood of an adult male is 15 ng/dL. The desired minimum level of free testosterone in the blood of an adult woman is 1.2 ng/dL.

Preferably, the desired minimum level of estradiol in the blood of an adult woman being treated during menopause is between 150 and 250 ng/L and progesterone between 15 and 25 μg/L.

The emulsifier in the present method can be any type of emulsifier. In a preferred form, the emulsifier is a phosphatidylcholine from lecithin, more preferably a phosphatidylcholine from soybean lecithin. An example of this is Phospholipon® 90 G, a brand name for a pure phosphatidylcholine from soybean lecithin stabilized with 0.1% ascorbyl palmitate from Phospholipid GmbH.

According to an embodiment, the first composition is emulsified for at least 15-25 minutes, preferably for at least 18-22 minutes, more preferably for about 20 minutes.

According to an embodiment of the present method, the polypropylene glycol and emulsifier are present in the first composition in a 1:1 ratio, in a 1:2 ratio, a 1:3 ratio, a 1:4 ratio, a 1:5 ratio, a 1:6 ratio, in a 1:7 ratio, a 1:8 ratio, a 1:9 ratio, a 1:10 ratio, a 2:1 ratio, a 3:1 ratio, a 4:1 ratio, a 5:1 ratio, a 6:1 ratio, a 7:1 ratio, an 8:1 ratio, a 9:1 ratio or a 10:1 ratio.

The liposomal preparation obtained according to the method of any of the above embodiments is especially suited for administration to a patient per os (PO) or for intravenous (IV) or transdermal administration.

The method may further comprise a second emulsification step in which the above emulsified composition based on water, propylene glycol, an emulsifier and a hormone to be encapsulated or an ester of such hormone, is added to a second composition and again emulsified. The second composition comprises glycerin, water and a polymer. Optionally, the same rotor-stator emulsifier is used for the emulsification in the second emulsification step as for the first emulsification step.

Again, the rotor-stator emulsifier with a stator provided with perforations with a diameter comprised between 0.8 and 5 mm, preferably with a smallest distance between the perforations of 0.5-1.5 mm, and preferably with a rotational speed of at least 6000 rpm, ensures that a liposomal preparation is obtained wherein, when administered, in this case topical/transdermal or per os administration, serum levels in the blood of the administered hormone are kept constant for a duration of at least 8-32 hrs. This prolonged release of encapsulated hormones also has the advantage that a single application per day can be sufficient.

The polymer of the second composition can be any type of polymer known to those skilled in the art, suitable for use in/for the production of such liposomal preparation.

According to an embodiment, the polymer of the second composition is a neutralized carbomer, such as a synthetic polymer of acrylic acid, i.e. a polyacrylic acid or carbomer. An example of this is Carbopol®, a brand name for types of carbomers. Carbomer is often used as an emulsifier and as a thickener to make a product spreadable. Carbomer is used, among other things, to form (hydro)gels, for example when used in hand gels or artificial tears. Carbomers are designated based on their molecular weight and specific constituents. For example, there are Carbomer 910, 934, 940, 941, 934P, 974P and 980.

Gel based on Carbomer 980, for example, is ideally suited for dermal use. This carbomer produces more transparent gels than the variety 974P recommended for buccal and oral use. For example, as a neutralizing ingredient trometamol can be used, which is preferred over triethanolamine (trolamine) which may contain secondary amines which, even after application to the skin, can convert to carcinogenic nitrosamines.

According to an embodiment, the emulsified composition of the first composition is emulsified together with the second composition for at least 10 to 20 minutes, preferably for at least 12 to 18 minutes, more preferably for about 15 minutes.

According to an embodiment of the present method, the polypropylene glycol and emulsifier present in the first composition and the glycerin present in the second composition are present in a 1:1:1 ratio, in a 1:1:1, 1:2:1, 1:3:1, 1:4:1, 1:5:1, 1:6:1, 1:7:1, 1:8:1, 1:9:1, 1:10:1, 2:1:1, 2:2:1, 2:3:1, 2:4:1, 2:5:1, 2:6:1, 2:7:1, 2:8:1, 2:9:1, 2:10:1, 3:1:1, 3:2:1, 3:3:1, 3:4:1, 3:5:1, 3:6:1, 3:7:1, 3:8:1, 3:9:1, 3:10:1, 4:1:1, 4:2:1, 4:3:1, 4:4:1, 4:5:1, 4:6:1, 4:7:1, 4:8:1, 4:9:1, 4:10:1 5:1:1, 5:2:1, 5:3:1, 5:4:1, 5:5:1, 5:6:1, 5:7:1, 5:8:1, 5:9:1, or 5:10:1 ratio, or in a 1:1:2, 1:2:2, 1:3:2, 1:4:2, 1:5:2, 1:6:2, 1:7:2, 1:8:2, 1:9:2, 1:10:2, 2:1:2, 2:2:2, 2:3:2, 2:4:2, 2:5:2, 2:6:2, 2:7:2, 2:8:2, 2:9:2, 2:10:2, 3:1:2, 3:2:2, 3:3:2, 3:4:2, 3:5:2, 3:6:2, 3:7:2, 3:8:2, 3:9:2, 3:10:2, 4:1:2, 4:2:2, 4:3:2, 4:4:2, 4:5:2, 4:6:2, 4:7:2, 4:8:2, 4:9:2, 4:10:2, 5:1:2, 5:2:2, 5:3:2, 5:4:2, 5:5:2, 5:6:2, 5:7:2, 5:8:2, 5:9:2, 5:10:2 ratio, or in a 1:1:3, 1:2:3, 1:3:3, 1:4:3, 1:5:3, 1:6:3, 1:7:3, 1:8:3, 1:9:3, 1:10:3, 2:1:3, 2:2:3, 2:3:3, 2:4:3, 2:5:3, 2:6:3, 2:7:3, 2:8:3, 2:9:3, 2:10:3, 3:1:3, 3:2:3, 3:3:3, 3:4:3, 3:5:3, 3:6:3, 3:7:3, 3:8:3, 3:9:3, 3:10:3, 4:1:3, 4:2:3, 4:3:3, 4:4:3, 4:5:3, 4:6:3, 4:7:3, 4:8:3, 4:9:3, 4:10:3, 5:1:3, 5:2:3, 5:3:3, 5:4:3, 5:5:3, 5:6:3, 5:7:3, 5:8:3, 5:9:3 or 5:10:3 ratio, or in a 1:1:4, 1:2:4, 1:3:4, 1:4:4, 1:5:4, 1:6:4, 1:7:4, 1:8:4, 1:9:4, 1:10:4, 2:1:4, 2:2:4, 2:3:4, 2:4:4, 2:5:4, 2:6:4, 2:7:4, 2:8:4, 2:9:4, 2:10:4, 3:1:4, 3:2:4, 3:3:4, 3:4:4, 3:5:4, 3:6:4, 3:7:4, 3:8:4, 3:9:4, 3:10:4, 4:1:4, 4:2:4, 4:3:4, 4:4:4, 4:5:4, 4:6:4, 4:7:4, 4:8:4, 4:9:4, 4:10:4, 5:1:4, 5:2:4, 5:3:4, 5:4:4, 5:5:4, 5:6:4, 5:7:4, 5:8:4, 5:9:4, 5:10:4 ratio, or in a 1:1:5, 1:2:5, 1:3:5, 1:4:5, 1:5:5, 1:6:5, 1:7:5, 1:8:5, 1:9:5, 1:10:5, 2:1:5, 2:2:5, 2:3:5, 2:4:5, 2:5:5, 2:6:5, 2:7:5, 2:8:5, 2:9:5, 2:10:5, 3:1:5, 3:2:5, 3:3:5, 3:4:5, 3:5:5, 3:6:5, 3:7:5, 3:8:5, 3:9:5, 3:10:5, 4:1:5, 4:2:5, 4:3:5, 4:4:5, 4:5:5, 4:6:5, 4:7:5, 4:8:5, 4:9:5, 4:10:5, 5:1:5, 5:2:5, 5:3:5, 5:4:5, 5:5:5, 5:6:5, 5:7:5, 5:8:5, 5:9:5 or 5:10:5 ratio.

The liposomes in the liposomal preparation produced according to the above method in any of the embodiments preferably have a diameter or particle size of 160 to 180 nm. The particle size can be measured by any of the methods known to those skilled in the art. For example, the particle size and distribution (see below) can be measured via a dynamic light scattering technique at 25° C. and presented as an intensity-weighted mean particle diameter (Z-mean).

According to an embodiment, at least 99%, at least 98%, at least 97%, at least 96%, at least 95%, at least 94%, at least 93%, at least 92%, at least 91%, at least 90%, at least 89%, at least 88%, at least 87%, at least 86%, at least 85%, at least 84%, at least 83%, at least 82%, at least 81%, at least 80%, at least 80% of the liposomes in the liposomal preparation produced according to the above method in any of the embodiments have a diameter or particle size of 160 to 180 nm.

The liposomal preparation obtained according to the method of any of the above embodiments in which the second emulsification step is included is extremely suitable for transdermal administration to a patient.

In a second aspect, the present invention relates to a liposomal preparation comprising liposomes with encapsulated hormones or esters thereof, wherein the preparation contains water, propylene glycol, an emulsifier and hormone to be encapsulated, wherein the liposomes have a single bilayer, and wherein at least 80% of the liposomes have a diameter or particle size which is comprised between 160 and 180 nm.

According to an embodiment, at least 99%, at least 98%, at least 97%, at least 96%, at least 95%, at least 94%, at least 93%, at least 92%, at least 91%, at least 90%, at least 89%, at least 88%, at least 87%, at least 86%, at least 85%, at least 84%, at least 83%, at least 82%, at least 81%, at least 80%, at least 80% of the liposomes in the liposomal preparation of the present invention have a diameter or particle size of 160 to 180 nm, measured as already described above.

The group of hormones encapsulated in the liposomes also includes esters of hormones and are preferably selected from sex hormones as already described above. The emulsifier is preferably as already described above.

According to an embodiment of the liposomal preparation, polypropylene glycol and emulsifier are present in a 1:1 ratio, in a 1:2 ratio, a 1:3 ratio, a 1:4 ratio, a 1:5 ratio, a 1:6 ratio, in a 1:7 ratio, a 1:8 ratio, a 1:9 ratio, a 1:10 ratio, a 2:1 ratio, a 3:1 ratio, a 4:1 ratio, a 5:1 ratio, a 6:1 ratio, a 7:1 ratio, an 8:1 ratio, a 9:1 ratio or a 10:1 ratio.

According to an embodiment, the liposomal preparation further comprises glycerin and a polymer, such as a neutralized carbomer. The polymer is preferably a polymer as already described above.

According to a further embodiment of the liposomal preparation, polypropylene glycol, emulsifier and glycerin are present in a 1:1:1 ratio, in a 1:1:1, 1:2:1, 1:3:1, 1:4:1, 1:5:1, 1:6:1, 1:7:1, 1:8:1, 1:9:1, 1:10:1, 2:1:1, 2:2:1, 2:3:1, 2:4:1, 2:5:1, 2:6:1, 2:7:1, 2:8:1, 2:9:1, 2:10:1, 3:1:1, 3:2:1, 3:3:1, 3:4:1, 3:5:1, 3:6:1, 3:7:1, 3:8:1, 3:9:1, 3:10:1, 4:1:1, 4:2:1, 4:3:1, 4:4:1, 4:5:1, 4:6:1, 4:7:1, 4:8:1, 4:9:1, 4:10:1 5:1:1, 5:2:1, 5:3:1, 5:4:1, 5:5:1, 5:6:1, 5:7:1, 5:8:1, 5:9:1, or 5:10:1 ratio, or in a 1:1:2, 1:2:2, 1:3:2, 1:4:2, 1:5:2, 1:6:2, 1:7:2, 1:8:2, 1:9:2, 1:10:2, 2:1:2, 2:2:2, 2:3:2, 2:4:2, 2:5:2, 2:6:2, 2:7:2, 2:8:2, 2:9:2, 2:10:2, 3:1:2, 3:2:2, 3:3:2, 3:4:2, 3:5:2, 3:6:2, 3:7:2, 3:8:2, 3:9:2, 3:10:2, 4:1:2, 4:2:2, 4:3:2, 4:4:2, 4:5:2, 4:6:2, 4:7:2, 4:8:2, 4:9:2, 4:10:2, 5:1:2, 5:2:2, 5:3:2, 5:4:2, 5:5:2, 5:6:2, 5:7:2, 5:8:2, 5:9:2, 5:10:2 ratio, or in a 1:1:3, 1:2:3, 1:3:3, 1:4:3, 1:5:3, 1:6:3, 1:7:3, 1:8:3, 1:9:3, 1:10:3, 2:1:3, 2:2:3, 2:3:3, 2:4:3, 2:5:3, 2:6:3, 2:7:3, 2:8:3, 2:9:3, 2:10:3, 3:1:3, 3:2:3, 3:3:3, 3:4:3, 3:5:3, 3:6:3, 3:7:3, 3:8:3, 3:9:3, 3:10:3, 4:1:3, 4:2:3, 4:3:3, 4:4:3, 4:5:3, 4:6:3, 4:7:3, 4:8:3, 4:9:3, 4:10:3, 5:1:3, 5:2:3, 5:3:3, 5:4:3, 5:5:3, 5:6:3, 5:7:3, 5:8:3, 5:9:3 or 5:10:3 ratio, or in a 1:1:4, 1:2:4, 1:3:4, 1:4:4, 1:5:4, 1:6:4, 1:7:4, 1:8:4, 1:9:4, 1:10:4, 2:1:4, 2:2:4, 2:3:4, 2:4:4, 2:5:4, 2:6:4, 2:7:4, 2:8:4, 2:9:4, 2:10:4, 3:1:4, 3:2:4, 3:3:4, 3:4:4, 3:5:4, 3:6:4, 3:7:4, 3:8:4, 3:9:4, 3:10:4, 4:1:4, 4:2:4, 4:3:4, 4:4:4, 4:5:4, 4:6:4, 4:7:4, 4:8:4, 4:9:4, 4:10:4, 5:1:4, 5:2:4, 5:3:4, 5:4:4, 5:5:4, 5:6:4, 5:7:4, 5:8:4, 5:9:4, 5:10:4 ratio, or in a 1:1:5, 1:2:5, 1:3:5, 1:4:5, 1:5:5, 1:6:5, 1:7:5, 1:8:5, 1:9:5, 1:10:5, 2:1:5, 2:2:5, 2:3:5, 2:4:5, 2:5:5, 2:6:5, 2:7:5, 2:8:5, 2:9:5, 2:10:5, 3:1:5, 3:2:5, 3:3:5, 3:4:5, 3:5:5, 3:6:5, 3:7:5, 3:8:5, 3:9:5, 3:10:5, 4:1:5, 4:2:5, 4:3:5, 4:4:5, 4:5:5, 4:6:5, 4:7:5, 4:8:5, 4:9:5, 4:10:5, 5:1:5, 5:2:5, 5:3:5, 5:4:5, 5:5:5, 5:6:5, 5:7:5, 5:8:5, 5:9:5 or 5:10:5 ratio.

The liposomal preparation can be used for administration either per os (PO) or intravenously (IV), or topically through the skin i.e. transdermally, for the purpose of sustained release of the encapsulated hormone into the blood stream. As a result, the blood serum levels of the hormone are relatively constant over a longer period of time, preferably at least 8 hrs, preferably at least 10 hrs, at least 12 hrs, at least 14 hrs, at least 16 hrs, at least 18 hrs, at least 20 hrs, at least 22 hrs, at least 24 hrs, at least 26 hrs, at least 28 hrs, at least 30 hrs, or at least 32 hrs, preferably for at least 8 to 32 hrs. By releasing the hormone for a long time and keeping the hormone in the bloodstream relatively constant for a long time, the number of applications or administrations of the hormone can be reduced. Preferably, one administration per day is sufficient to obtain and maintain a relatively constant blood serum level. In another example, 2 or 3 administrations per day are required to obtain and maintain a relatively constant blood serum level.

According to an embodiment, the liposomal preparation of the present invention does not comprise organic solvents such as alcohols including ethanol, isopropanol and methanol. An advantage of this is that, due to the absence of such alcohols, the preparation is much more skin-friendly and prevents the skin at the application site from drying out and becoming damaged during transdermal administration, especially after multiple administrations of the preparation with encapsulated hormone, for example via a daily topical application. In addition, methanol, for example, is very toxic when administered per os. When administered per os it is very important that the preparation does not contain methanol. According to another or further embodiment, the liposomal preparation comprises solvents selected from water, propylene glycol and glycerin. Both propylene glycol and glycerin have a moisturizing effect on the skin, unlike the above alcohols such as ethanol, isopropanol and methanol.

According to an embodiment, the liposomal preparation of the present invention as described in any of the preceding embodiments is obtained by the method of the present invention as described in any of the preceding embodiments.

According to an embodiment, the liposomes of the liposomal preparation have an entrapment efficiency of at least 95%, preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, more preferably at least 99.9%.

Entrapment efficiency (EE), also known as encapsulation efficiency, refers to the liposomal loading capacity. The greater the EE, the greater the amount of drug that can be encapsulated and transported to the target tissues.

The % EE is calculated using the following equation:

$$EE \% = \frac{De - Df}{De} \times 100 \%$$

where De is the total amount of testosterone in the liposomes and Df is the amount of free drug (testosterone) in the filtrate. Another way to express the above equation is:

EE(%)=(amount of drug in liposomes×100)/amount of drug used to make liposomes.

An EE of at least 95% is extremely high, making the liposomal preparation extremely efficient in incorporating/encapsulating the hormones into the liposomes, which when administered to a patient can transfer these hormones to the patient.

In a third aspect, the present invention relates to a liposomal preparation for use in hormone therapy or for use in the treatment of a patient requiring hormone therapy. In a fourth aspect, the present invention relates to a liposomal preparation for use in the treatment of hypogonadism. In a fifth aspect, the present invention relates to a liposomal preparation for use in the treatment of adrenal insufficiency. For the third, fourth and fifth aspects, the liposomal preparation as described in any of the preceding embodiments, is preferably produced by a method as described above in any of the preceding embodiments.

“Hormone therapy” or “hormonal therapy” refers to any type of therapy that uses hormones in medical treatments. Treatment with hormone antagonists may also be referred to as hormonal therapy or anti-hormone therapy. The most common classes of hormone therapy are oncological hormone therapy, hormone replacement therapy (for menopause), androgen replacement therapy (ART, including testosterone replacement therapy (TRT)), oral contraceptive pills, and transgender hormone therapy. Further types of hormone therapy have already been described above.

Hypogonadism is a condition in which there is reduced functional activity of the gonads—the testes or ovaries—which can lead to decreased production of sex hormones. Low levels of androgen (e.g., testosterone) are referred to as hypoandrogenism and low levels of estrogen (e.g., estradiol) are referred to as hypoestrogenism. In addition, this is also linked to abnormally low levels of progestogens such as progesterone in women.

Hypogonadism, commonly referred to by the symptom of “low testosterone levels” or “low T levels,” can also lead to a decrease in other hormones secreted by the gonads, such as progesterone, DHEA, anti-Müllerian hormone, activin, and inhibin. The development of the sperm (spermatogenesis) and the release of the egg cell from the ovaries (ovulation) can be impaired by hypogonadism, which, depending on the severity of the condition, can lead to partial or complete infertility.

Symptoms of hypogonadism include erectile dysfunction, decreased sexual desire, fatigue, loss of energy, depressive mood, impaired cognition, regression of secondary sexual characteristics, or osteoporosis, regardless of the underlying etiology. Examples of such conditions are:
Primary (hypergonadotropic) hypogonadism (congenital or acquired): e.g., testicular failure due to cryptorchidism, bilateral torsion, orchitis, disappearing testis syndrome, orchiectomy, Klinefelter's syndrome, chemotherapy, or toxic damage from alcohol or heavy metals. Men with primary hypogonadism usually have low serum testosterone levels, and gonadotropins (follicle-stimulating hormone [FSH], luteinizing hormone [LH]) above the normal range. The normal range of LH and FSH is between 2 and 5 mIU/ml. Higher values indicate hypogonadism, while lower values indicate pituitary problems or an inhibition of the feedback system due to overdose.
Secondary (hypogonadotropic) hypogonadism (congenital or acquired): e.g., idiopathic deficiency of gonadotropin or luteinizing hormone-releasing hormone (LHRH) or pituitary-hypothalamus injury from tumors, trauma or radiation. Men with secondary hypogonadism have low serum testosterone levels (less than 8.5-10.2 nmol/L or 250-300 ng/dL) but have gonadotropins below or within the normal range (2-5 mIU/ml for LH and FSH).

For example, a low testosterone level can be a result of a testicular carcinoma, a varicocele, a hydrocele, a cryptorchidism, pituitary failure, older age or an iatrogenic cause such as sterilization.

For example, a low estrogen level can be a result of menopause, pituitary failure, polycystic ovaries (PCO) or a genetic abnormality that which causes the ovaries to be absent/underdeveloped.

Low progesterone levels can also be a result of menopause, pituitary failure, or a genetic abnormality which causes the ovaries to be absent/underdeveloped.

“Adrenal insufficiency” or “adrenocortical insufficiency” is a condition in which the adrenal glands do not produce or produce insufficient amounts of the corticosteroid hormones, especially the stress hormone cortisol, but the production of aldosterone (a mineralocorticoid) that regulates sodium, potassium and water retention, may also be disrupted. Cravings for salt or salty dishes due to the loss of sodium in the urine from aldosterone deficiency is common. There are three types of adrenocortical insufficiency. First, primary adrenocortical insufficiency due to adrenal impairment. This is called Addison's disease. Among these, 80% are due to autoimmune disease or autoimmune adrenalitis. Other cases are due to adrenogenital syndrome or an adenoma (benign tumor) of the adrenal gland. The disease is called idiopathic if there is no known cause of the adrenocortical insufficiency. Secondary adrenal insufficiency is caused by impairment of the pituitary gland or hypothalamus. This may be due to an adenoma such as a pituitary microadenoma or a hypothalamic tumor, due to Sheehan's syndrome, which is associated with a reduction of the pituitary gland alone, or due to a past head injury. Tertiary adrenal insufficiency is due to hypothalamic disease and reduction of corticotropin releasing factor (CRF).

It will also be apparent to one skilled in the art that the present invention also relates to a liposomal preparation according to any of the above embodiments, for use in the treatment of conditions associated with a decreased androgen level, a decreased estrogen level, or a decreased progestogen level such as a decreased progesterone level.

It will be apparent to a person skilled in the art that the present invention also relates to a liposomal preparation according to any of the above embodiments, for use in the treatment of conditions associated with a reduced cortisol level or aldosterone level. The present invention also relates to a liposomal preparation according to any of the above embodiments, for use in the treatment of Addison's disease, in the treatment of adrenogenital syndrome, in the treatment of adenoma of the adrenal gland, in the treatment of a pituitary microadenoma or a hypothalamic tumor, in the treatment of Sheehan's Syndrome and/or in the treatment of hypothalamic disease.

The liposomal preparation for use in any of the preceding aspects is preferably administered to a patient per os (PO), by intravenous administration (IV) or by transdermal administration. The liposomal preparation for transdermal administration was preferably produced by the method of the present invention, wherein both a first and a second emulsification step are present.

According to an embodiment, a single administration is required. In another embodiment, more than one administration is required.

For example, a single administration per day is sufficient. In another example, several administrations per day are required, for example two, three, four or five administrations per day. In yet another example, one administration every two, every three, every four, every five, every six or every seven days is required. In yet another example, one administration every 10 days, every two weeks, every three weeks, every month is required.

The present invention also relates to method for treating a patient requiring hormone therapy, wherein a liposomal preparation is administered according to any of the preceding embodiments; a method for treating hypogonadism, wherein a liposomal preparation is administered according to any of the preceding embodiments; and a method for treating adrenal insufficiency, wherein a liposomal preparation is administered according to any of the preceding embodiments.

In addition, the present invention also relates to a method for producing a drug for hormone therapy, for treating a patient requiring hormone therapy, for treating hypogonadism or for treating adrenal insufficiency, the drug comprising a liposomal preparation according to any of the preceding embodiments.

In what follows, the invention is described by way of non-limiting examples illustrating the invention, and which are not intended to and should not be interpreted as limiting the scope of the invention. It will also be appreciated that although the following examples relate to a liposomal preparation with encapsulated testosterone, similar experiments can be set up with other encapsulated hormones, preferably other encapsulated sex hormones as already discussed above.

### EXAMPLES

Example 1: Influence of the size of the perforations of the stator of a rotor-stator emulsifier on obtained serum levels in the blood of an encapsulated hormone In the present example, a liposomal preparation is prepared both by a method of the present invention (optimized method) and by a conventional method (conventional method). Subsequently, the obtained liposomal preparation was applied topically to the arm of a test subject (transdermal administration). In addition, Testarzon®, a commercially available non-liposomal, transdermal alcoholic gel with a concentration of mg testosterone per gram of gel was also tested, and the blood serum levels of the encapsulated hormone, testosterone in the current experiment, were compared at multiple time points.

Optimized method: the experimental stator used has perforations with a diameter of 1.6 mm where the distance between the perforations is 1 mm. The particle size of the obtained liposomes shows only one peak of particles with particle sizes between 160 and 180 nm.

Conventional method: The conventional stator used has one row of six perforations, the perforations having a diameter of 9 mm, and the distance between the perforations being 6 mm. The particle size of the obtained liposomes shows a peak at 250 nm, at 1900 nm and at 4500 nm.

Particle sizes were reported as intensity-weighted mean particle diameter (Z-mean) and were determined by dynamic light scattering technique on a Zetasizer Nano-ZS90 (Malvern Instruments, Malvern, UK). For this purpose, the liposomal preparations were diluted with water for injection to a suitable concentration for the measurement. In some cases, the dilution was filtered through a 5 μm cellulose acetate syringe filter (Sartorius). The measurements were performed at 25° C.

### a) Preparation of a 2.5% Testosterone-Containing Liposomal Preparation

For 100 ml liposomal preparation:

### Composition 1:

| | | |
|---|---|---|
| Propylene glycol: | 10 | ml |
| Phospholipon ®90G: | 10 | ml |
| Aqua purificata: | 64.85 | ml |

Testosterone propionate: 2.5 mg

### Composition 2:

| | | |
|---|---|---|
| Glycerin: | 10 | ml |
| Carbopol ®980 2%: | 2 | ml |
| Aqua conservans conc.: | 0.65 | ml |

Propylene glycol and water are heated to 70° C. in separate containers. The phospholipon 90G is added to the propylene glycol and allowed to melt in it. Then testosterone propionate is added to it, and then the water is added to it. This initial composition is emulsified a first time for 20 min in a rotor-stator emulsifier with a chosen perforated stator (either the experimental stator or the conventional stator) at a high speed of at least 6000 rpm.

Subsequently, carbopol is mixed with glycerin and aqua conservans and added to the emulsified mixture, after which the mixture is emulsified a second time for 15 minutes in the rotor-stator emulsifier with a chosen perforated stator (either the experimental stator or the conventional stator) at a high speed of at least 6000 rpm. The obtained 2.5% testosterone-containing liposomal preparation is now ready for topical application to the skin, with the aim of obtaining a transdermal transport of testosterone into the blood of a test subject.

### b) Administration of the 2.5% Testosterone-Containing Liposomal Preparation or Testarzon® to a Test Subject

A menopausal woman weighing 52 kg was chosen as the test subject, who does not have endogenous testosterone production that male test subjects do have and therefore has no circadian rhythm for testosterone production. The measured serum levels in the blood are therefore exclusively due to the testosterone present in the 2.5% testosterone-containing liposomal preparation or in Testarzon®.

The metabolization of testosterone to androstandiol glucuronide was minimized by the test subject's intake of 5 mg of finasteride. The test subject applied the testosterone-containing liposomal preparation to the inside of the arms in an amount of 1 ml of a liposomal preparation containing 2.5% testosterone, the equivalent of 25 mg testosterone; or 1.25 g of Testarzon® at a concentration of 20 mg of testosterone per gram of transdermal gel, also the equivalent of 25 mg of testosterone.

Administration was done two days in a row at 9 am. Blood samples were taken from 1 hr after administration at 1 hr intervals.

### c) Measured Results

The measurements of the blood samples are shown in the tables below, in which:
dehydroepiandrosterone (DHEA) is expressed in μg/dL,
total testosterone (Testosterone) is expressed in ng/dL,
sex hormone-binding globulin (SHBG) is expressed in nmol/L,
free testosterone (Free Testo) is expressed in ng/dL, and
androstandiol glucuronide (Andro-gluc) is expressed in ng/dL.

DHEA is a testosterone precursor;

SHBG is a glycoprotein that is a binding protein, also called carrier protein or carrier, that binds to androgens (testosterone, dihydrotestosterone and DHEA) and to estrogens.

Test I: 1 ml Liposomal Preparation of 2.5% Testosterone Prepared with an Experimental Stator with 1.6 mm Perforations, the Equivalent of 25 mg Testosterone

Day 1 liposomal preparation of 2.5% testosterone; transdermal administration at 9 am followed by a blood draw every hour between 10 am and 5 pm.

| | | | | | | | | | |
|---|---|---|---|---|---|---|---|---|---|
| | | | | 12 | | | | | |
| Hormones | 9am | 10am | 11am | noon | 1pm | 2pm | 3pm | 4pm | 5pm |

| | | | | | | | | |
|---|---|---|---|---|---|---|---|---|
| DHEA | 105 | 106 | 104 | 111 | 111 | 109 | 105 | 104 |
| Testosterone | 8 | 16 | 29 | 38 | 43 | 63 | 71 | 90 |
| SHBG | 97 | 99.9 | 99.1 | 99.6 | 103.1 | 107.5 | 104.4 | 105.3 |
| Free Testo | ≤0.10 | 0.13 | 0.23 | 0.31 | 0.34 | 0.47 | 0.55 | 0.69 |
| Andro-gluc. | 0.86 | 0.52 | 0.82 | 0.85 | 1.99 | 0.73 | 0.47 | 0.63 |

Day 2 liposomal preparation of 2.5% testosterone; transdermal administration at 9 am followed by a blood draw every hour between 10 am and 5 pm. The values of the 9 am blood draw are the result of the application of day 1 at 9 am.

| | | | | 12 | | | | | |
|---|---|---|---|---|---|---|---|---|---|
| Hormones | 9am | 10am | 11am | noon | 1pm | 2pm | 3pm | 4pm | 5pm |
| DHEA | 124 | 122 | 125 | 128 | 122 | 132 | 141 | 142 | 125 |
| Testosterone | 102 | 158 | 166 | 197 | 214 | 227 | 214 | 269 | 267 |
| SHBG | 90 | 92.2 | 93.8 | 98.8 | 95.8 | 98.8 | 100.3 | 97 | 89.9 |
| Free Testo | 0.93 | 1.42 | 1.47 | 1.67 | 1.87 | 1.95 | 1.8 | 2.36 | 2.53 |
| Andro-gluc. | 2.74 | 1.69 | 1.32 | 0.89 | 1.21 | 0.86 | 1.2 | 1.35 | 1.29 |

Test II: 1.25 g Testarzon® with a Concentration of 20 mg Testosterone Per Gram of Transdermal Gel, the Equivalent of 25 mg Testosterone.

Testarzon® is a commercially available transdermal alcoholic gel with a concentration of 20 mg testosterone per gram of transdermal gel but is not a liposomal preparation. Test I was repeated but now with Testarzon® at a dose equivalent to 25 mg of testosterone, or 1.25 g from a pump of non-liposomal alcoholic gel, applied by the same test subject but after a 10-day rest interval after the previous Test I.

Day 1 Testarzon® with 25 mg testosterone; transdermal administration at 9 am followed by a blood draw every hour between 10 am and 5 pm.

| | | | | | | | | | |
|---|---|---|---|---|---|---|---|---|---|
| | | | | 12 | | | | | |
| Hormones | 9am | 10am | 11am | noon | 1pm | 2pm | 3pm | 4pm | 5pm |

| | | | | | | | | |
|---|---|---|---|---|---|---|---|---|
| DHEA | 126 | 124 | 131 | 136 | 128 | 134 | 132 | 136 |
| Testosterone | 44 | 64 | 114 | 109 | 121 | 151 | 131 | 129 |
| SHBG | 87.2 | 87.9 | 88.6 | 93.8 | 91.5 | 95.7 | 94.6 | 100.9 |
| Free Testo | 0.4 | 0.59 | 1.05 | 0.95 | 1.08 | 1.31 | 1.14 | 1.06 |
| Andro-gluc. | 0.99 | 0.5 | 0.43 | 0.7 | 1.07 | 0.89 | 0.72 | 0.42 |

Day 2 Testarzon® with 25 mg testosterone; transdermal administration at 9 am followed by a blood draw every hour between 10 am and 5 pm. The values of the 9 am blood draw are the result of the application of day 1 at 9 am.

The application of the second day was done 1 hr before the first blood draw with the same amount of Testarzon®. The values of the 9 am blood draw are the result of the application of day 1 at 9 am.

| | | | | 12 | | | | | |
|---|---|---|---|---|---|---|---|---|---|
| Hormones | 9am | 10am | 11am | noon | 1pm | 2pm | 3pm | 4pm | 5pm |
| DHEA | 125 | / | 138 | 150 | 151 | 150 | 156 | 157 | 151 |
| Testosterone | 192 | 256 | 198 | 241 | 225 | 212 | 198 | 197 | 180 |
| SHBG | 99.8 | 105.8 | 106.1 | 104.8 | 107.2 | 107.5 | 109.1 | 109.4 | 112.4 |
| Free Testo | 1.62 | 2.07 | 1.58 | 1.97 | 1.77 | 1.68 | 1.54 | 1.52 | 1.35 |
| Andro-gluc. | / | / | / | / | / | / | / | / | / |

Test III: 1 ml Liposomal Preparation of 2.5% Testosterone Prepared with an Experimental Stator with 9 mm Perforations, the Equivalent of 25 mg Testosterone

Test I was repeated but this time with 1 ml of a liposomal preparation of 2.5% testosterone, a dose equivalent to 25 mg of testosterone, applied by the same test subject but after an interval of 10 days of rest after the previous Test II.

Day 1 liposomal preparation of 2.5% testosterone; transdermal administration at 9 am followed by a blood draw every hour between 10 am and 5 pm.

| | | | | | | | | | |
|---|---|---|---|---|---|---|---|---|---|
| | | | | 12 | | | | | |
| Hormones | 9am | 10am | 11am | noon | 1pm | 2pm | 3pm | 4pm | 5pm |

| | | | | | | | | |
|---|---|---|---|---|---|---|---|---|
| DHEA | 161 | 169 | 174 | 176 | 185 | 188 | 189 | 193 |
| Testosterone | 19 | 21 | 28 | 32 | 35 | 40 | 37 | 37 |
| SHBG | 57.3 | 57.9 | 57.6 | 56.9 | 57.9 | 58.9 | 59.2 | 59.1 |
| Free Testo | 0.23 | 0.25 | 0.34 | 0.39 | 0.42 | 0.47 | 0.44 | 0.44 |
| Andro-gluc. | 1.09 | 0.43 | 0.85 | 0.56 | 0.79 | 0.56 | 0.62 | 0.79 |

For Test III, no results were measured on a second day as the test results of day 1 were so substandard that a second day was not useful.

### d) Comparison and Interpretation of Test Results

Below, the test results of the liposomal preparation of 2.5% according to the invention, prepared with an experimental stator, are compared with the test results of the commercially available non-liposomal transdermal testosterone gel Testarzon®.

A different but high serum level after 32 hrs at the end of day 2 is observed for the liposomal preparation (LG) according to the invention and for the non-liposomal gel (TZ) of Testarzon®, i.e.
total testosterone: 267 ng/dL (LG) and 180 ng/dL (TZ)
free testosterone: 2.53 ng/dL (LG) and 1.35 ng/dL (TZ).

Comparing the serum level after 8 hrs at the end of day 1 when the liposomal preparation prepared with the experimental stator (LGexp) was administered with the liposomal preparation prepared with the conventional stator (LGcon), it was found that 8 hrs after administration the following serum levels were measured:
total testosterone: 90 ng/dL (LGexp) and 37 ng/dL (LGcon)
free testosterone: 0.69 ng/dL (LGexp) and 0.44 ng/dL (LGcon).

This shows that using the liposomal preparation produced by the method using the experimental stator, the serum levels obtained 8 hrs after administration are almost three times higher for total testosterone and almost two times higher for free testosterone than with a liposomal preparation prepared with a conventional stator.

If the serum level is compared after 32 hrs at the end of day two when administering the liposomal preparation prepared with the experimental stator (LGexp) with non-liposomal alcoholic gel Testarzon® (TZ), it appears that the following serum levels are measured 32 hrs after administration:
total testosterone: 267 ng/dL (LGexp) and 180 ng/dL (TZ)
free testosterone: 2.53 ng/dL (LGexp) and 1.35 ng/dL (TZ)

This shows that the serum level obtained 32 hrs after administration is almost one and a half times higher for total testosterone and almost twice higher for free testosterone than with the non-liposomal gel Testarzon®.

However, using a conventional stator with 6 orifices of 9 mm to prepare the liposomal 2.5% gel, serum concentrations comparable to those of the non-liposomal testosterone gel Testarzon® cannot be achieved as early as 8 hrs at the end of day 1.

The following serum levels after 8 hrs at the end of day 1 were determined for the liposomal gel with conventional stator (CS) and for the non-liposomal gel (TZ) of Testarzon®:
total testosterone: 37 ng/dL (CS) and 129 ng/dL (TZ)
free testosterone: 0.44 ng/dL (CS) and 1.06 ng/dL (TZ)

This shows that the conventional stator does not allow the serum concentrations of the non-liposomal gel Testarzon® to be reached, and this is already apparent after the first day of transdermal administration.

With the experimental stator with 1.6 mm perforations according to the invention, the serum concentrations of the non-liposomal gel Testarzon® can be surpassed, so that the present invention offers the possibility to replace a transdermal non-liposomal alcoholic gel with a transdermal liposomal preparation that is alcohol-free. An advantage of this replacement is that the treatment is much more skin-friendly due to the absence of alcohol and prevents the skin on the arms from drying out and becoming damaged after multiple administrations of the desired hormone, in this case testosterone, via a daily topical application.

The measurements also show that a higher serum steady state concentration of testosterone and free testosterone can be obtained with this experimental stator, which implies that:
the liposomes show a high entrapment efficiency;
the liposomes show a high skin penetration;
only a small residue remains on the skin after 30 minutes with a low risk of contamination, in particular a low risk of transferring the hormone to a third party;
there is prolonged release of the hormone by the liposomes for at least 32 hrs;
that the therapeutically important free testosterone concentration in the blood after 32 hrs is almost double when using the liposomal preparation (2.53 ng/L (LG)) compared to the alcoholic gel Testarzon® (1.35 ng/L (TZ)). This difference is the result of at least two factors: first, the higher total concentration of total testosterone, and second, the lower concentration of SHBG. It is scientifically established that the higher the total testosterone concentration, the lower the SHBG concentration becomes. The action of the liposomes obtained with the method according to the invention is very simple and as follows. The liposomal preparation can be administered topically for transdermal administration, where the lipid bilayer is able to integrate into and between the cell membranes of the skin cells, so that the active ingredients encapsulated in the liposome are slowly but prolongedly released into the skin, or even transdermally through the skin and into the bloodstream. The liposomal preparation may also be administered per os (PO) or intravenously (IV).

In summary, using the experimental stator to produce a liposomal preparation with encapsulated hormone offers the following therapeutic advantages:
a single dose per day may be sufficient, which promotes treatment compliance and is cost effective;
possibility to accurately dose the hormone to 0.1 ml with a syringe given the high fluidity of the product obtained;
minimal risk of contamination, i.e. involuntary transfer to a third person;
a stable and high concentration of free testosterone and total testosterone is already reached in the blood after 32 hrs.

### Example 2: Figure Description of Several Rotor-Stator Emulsifiers as Described in Example 1

With the intention of better demonstrating the features of the invention, a preferred application of the method for producing a liposomal preparation according to the invention is described herein by way of example without any limiting character, with reference to the accompanying figures.

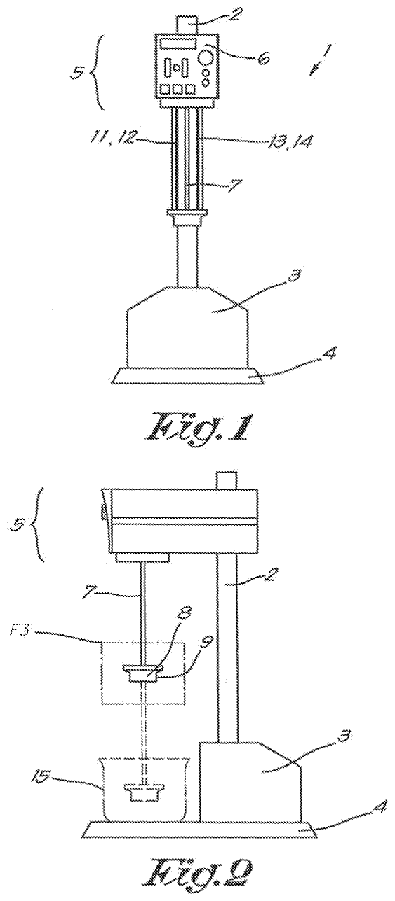

FIG. 1 shows schematically and in front view a rotor-stator emulsifier 1 used in the method for producing a liposomal preparation according to an embodiment of the invention, consisting of a stand 2 mounted on a base 3 placed on a support plate 4, and a housing 5 with motor and control panel 6 with control buttons to control the drive speed and rotation time of a centrally mounted drive shaft 7. The drive shaft 7 connects the motor in the housing 5 to a rotor 8 being an impeller that rotates within a stator 9 being a static ring with perforations 10 located around the impeller in a fixed position. The stator 9 is held in its fixed position by four stationary support rods 11, 12, 13, 14 fixed to the housing 5 with motor in a fixed position.

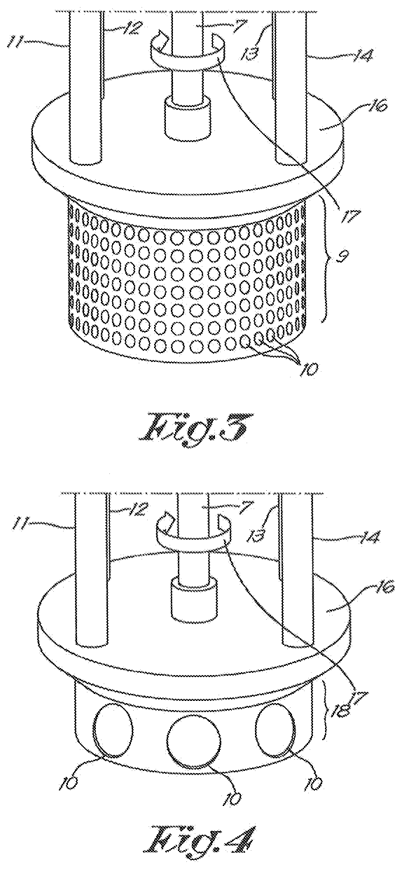

FIG. 2 shows a side view of FIG. 1, showing how the drive shaft 7 with rotor 8 and stator 9 can be lowered by sliding the housing 5 over the stand 2, so that the rotor 8 and stator 9 are placed in a receptacle 15 filled with the mixture to be emulsified.

FIG. 3 shows in more detail an experimental stator 9 with 1.6 mm perforations mounted on a fixed disk 16 held in its fixed position by four stationary support rods 11, 12, 13, 14. The experimental stator 9 is provided with perforations 10 with a diameter of 1.6 mm with a mutual distance of 1 mm, which perforations are arranged one below the other in horizontal rows. The mixture to be emulsified is forced through the perforations 10, by an impeller driven by the central drive shaft 7. The direction of rotation of the drive shaft 7 is indicated by the arrow 17.

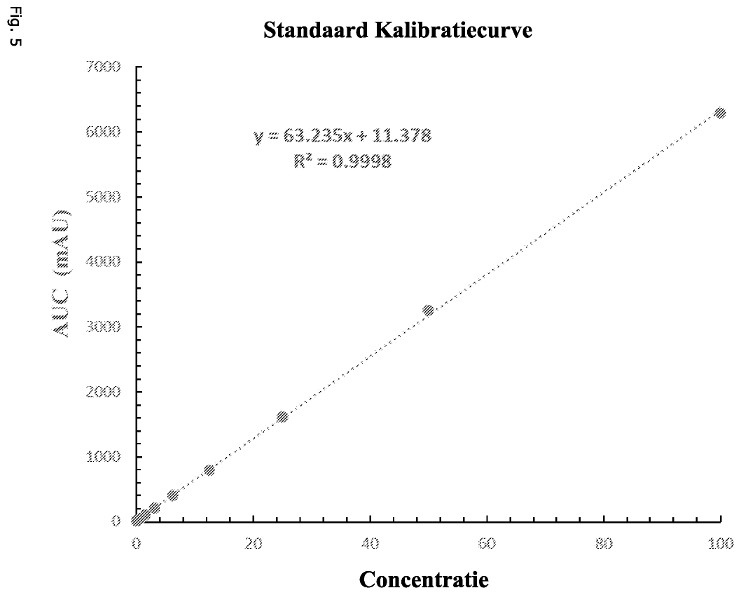

FIG. 4 shows in more detail a conventional stator 18 with six 9 mm diameter perforations 10 mounted on a fixed disk 16 held in its fixed position by four stationary support rods 11, 12, 13, 14. The conventional stator 18 is provided with six perforations 10 with a diameter of 9 mm and a distance of 6 mm, which together form one horizontal row on the conventional stator 18. The mixture to be emulsified is forced through the six perforations 10, by an impeller driven by the central drive shaft 7. The direction of rotation of the drive shaft 7 is indicated by the arrow 17.

The present invention is by no means limited to the embodiment described as an example and shown in the figures, but a liposomal preparation prepared according to any of the embodiments of the method of the invention can be realized in all kinds of embodiments and dimensions without departing from the scope of the invention, as defined in the claims below.

### Example 3: In Vitro Skin Permeation Analysis

Examples 3 to 8 represent several successive parts of the same large experiment. However, this does not exclude the fact that some of these parts could be performed in a different chronological order.

Example 3 concerns a comparative analysis of the skin permeation profile between the liposomal preparation with encapsulated testosterone of the present invention obtained by a method of the present invention and one of reference products Testarzon® or Androgel®, which are available on the commercial market. For this purpose, in vitro skin permeation testing (IVPT) and tests for deposition in the skin are performed.

The purpose of this is to develop a complete comparison of the skin permeation profile between the liposomal preparation of the present invention and the reference product available on the market by performing in vitro skin permeation testing (IVPT) and an analysis of the drug content in the skin. To achieve the objectives, we will first develop and validate a high performance liquid chromatography (HPLC) assay for testosterone (drug in both formulations) in both buffer samples and skin extraction samples (Task 1.1), followed by IVPT experiments (Task 1.2) and testosterone skin retention analyses (Task 1.3).

### Task 1.1: Development and Validation of a Testosterone HPLC Assay

Many HPLC methods for testosterone have been reported in the literature, including quantitation in dosage forms such as capsules as well as in patient serum samples. In the current experiment, the methods reported specifically for IVPT are chosen, in particular the following assay is selected:

Isocratic HPLC is used with a Phenomenex C18 column, 5 μm, 50 mm×4.6 mm at 25° C. UV detection occurs at 241 nm with a UV/Vis detector in an Agilent 1100 HPLC system with ChemStation software. A degassed mixture 60:40 of acetonitrile and water with a flow rate of 1 ml/min is used as mobile phase. Testosterone is detected at a retention time of approximately 1.3 min and the limit of the test is expected to be approximately 0.1 μg/ml with a coefficient of variation (CV) of approximately 0.24%. The lower limit of detection and quantification (LOD and LOQ) is determined, as well as inter- and intra-day variability. Unknown testosterone concentrations in the samples of the IVPT tests are calibrated against known standards.

### Task 1.1 Thus Provides a Validated HPLC Analysis Method as a Result.

The result of this is as follows:
A degassed mixture 60:40 of acetonitrile and water with a flow rate of 0.5 ml/min (instead of 1 ml/min) was used as the mobile phase. Testosterone was detected at a retention time of approximately 4.4 min.

An injection volume of 10 μl was used and the duration was 10 min. The lower limit of detection (LOD) was 0.53 μg/ml and the lower limit of quantification (LOQ) was 1.62 μg/ml, with a coefficient of variation of approximately 0.74%.

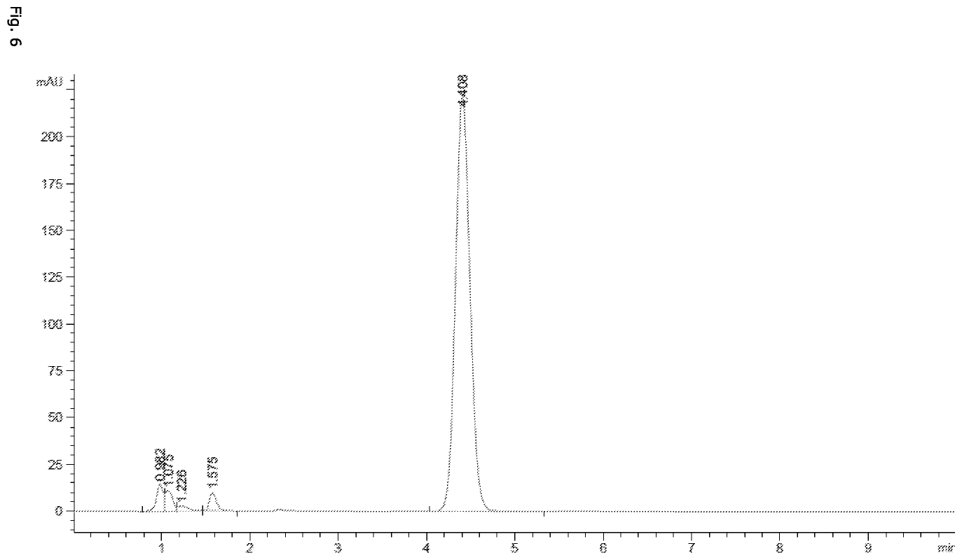

The resulting standard calibration curve is shown in FIG. 5: y=63.235x+11.378, R²=0.9998 (FIG. 5).

FIG. 6 represents an HPLC result of a tested sample (50 μg/ml testosterone+a skin extract solution) using the developed validation method at a flow rate of 0.5 ml/min. The graph shows the absorbance (mAU) as a function of time (min). A peak corresponding to the sample is shown at 4.408 min (retention time).

### Task 1.2: In Vitro Skin Permeation Testing (IVPT)

Human cadaver skin samples are obtained from recognized tissue banks in the US from Caucasian males aged 20-70 years, from the back of the body and dermatomed to 500 μm. The skin is stored at −80° C. and kept frozen for no more than a month before use. The skin samples are then thawed, cut to size and mounted in upper stratum corneum in vertical static glass Franz diffusion cells with a donor area of 0.64 cm² and a receptor volume of 5.1 ml. A minimum of 20 cells are used per group (reference formulation and test preparation formulation of the present invention), with a total of 40 cells. The cells are placed in a 32° C. heating block and equilibrated for 30 minutes. The receptor contains a suitable buffer such as phosphate buffered saline and is stirred with magnetic stirrers. The formulations to be tested on each skin are applied at t=0 and samples (300 μl) are taken from the receptor compartment of the Franz cell at predetermined time points over a total of 36 hours. The volume of the sample is immediately replaced with an equal volume of receptor buffer. The samples are filtered with 0.45 μm Millipore (Nalgene) filters before being injected into the HPLC equipment. All samples are analyzed using the validated HPLC assay developed in Task 1.1.

The permeation data is expressed as the cumulative amount of testosterone transported per unit of area across the skin membranes, as a function of time. Each permeation curve is fitted to the correct solution of Fick's law, assuming that a) there is no depletion of the drug in the donor compartment for the duration of the experiment, b) that the receptor compartment provides “sink” conditions, and c) that at t=0 no drug is present in the skin. The cumulative amount of drug permeating per unit of area is calculated using Equation 1:

$${Q}_{n} = \frac{{C}_{n} {V}_{r} + {\sum }_{i = 0}^{n - 1} {C}_{i} {V}_{s}}{A} \qquad ( 1 )$$

Herein, Qn is the cumulative amount of drug permeating per unit of area (μg/cm²) at different sampling times, Cn is the drug concentration in the receiving medium at the different sampling times, Ci is the drug concentration in the recipient medium at the i-th (n−1) sampling time, Vr is the volume of the receptor solution, Vs is the volume of the sample taken, and A is the effective permeation area of the diffusion cell. The Qn values are plotted against time and then the steady-state flux (Jss) is calculated from the slope of the linear portion of the graph.

The permeability coefficient (Kp) is calculated using equation 2:

$${K}_{p} = \frac{{Y}_{ss}}{{C}_{0}} \qquad ( 2 )$$

where C0 is the initial concentration of testosterone in the donor compartment. Task 1.2 thus results in a quantification of amounts/concentrations of testosterone transported across the skin membranes.

### Task 1.3: In Vitro Study of Drug Deposition in the Skin

At the end of the experiment (36 hours), the skin is harvested and separated into epidermis and dermis and extracted for testosterone. The separation of the layers is achieved by the length of the experiment, and the epidermis and dermis can be gently peeled apart with surgical tweezers. The extraction is validated and performed with a combination of organic solvents. All skin extraction samples are analyzed with the validated HPLC assay developed in Task 1.1.

Task 1.3 Thus Results in a Quantification of the Amounts/Concentrations of Testosterone Extracted from the Dermis and Epidermis.

Statistical significance is determined using ANOVA, while the Student's t-test is used to determine significant differences between the test samples and the reference. Differences are considered statistically significant when p<0.05. The results are expressed in means t standard deviations (SD).

### Example 4: Characterization of the Liposomal Preparation of the Present Invention Obtained by a Method of the Present Invention, with Respect to Liposome Particle Size Distribution, Zeta Potential, Particle Morphology, Testosterone Loading, Entrapment Efficiency, and In Vitro Release (IVRT) of the Testosterone

The main objective of this experiment is to develop a new liposomal formula (Task 2.1-2) and fully characterize the impact of formula and process design. In Task 2.3, the impact of formula and process parameters on liposome particle size and distribution is investigated, as well as the zeta potential. The data provides information on the uniformity of the liposomal particle size within the liposomal preparation and also the values of the charge on the liposomal vesicles (if any). In addition, particle morphology, drug loading, and entrapment efficiency are examined in Task 2.4. Task 2.5 investigates the in vitro release (IVRT) of testosterone from pilot formulas. This data shows the consistency of the testosterone release of the preparation of the present invention as well as how the amounts of the drug relate to time. These sampling times are performed until a release plateau is reached, which indicates the maximum amount of drug that the preparation of the present invention can release from its preparation microenvironment. Finally, a rheological study is performed as Task 2.6.

### Task 2.1: Pre-Formulation Data Regarding Optimal Excipients and Additives/Compatibility Analysis

Liposome formulas mainly consist of the active ingredient and phospholipids, but also contain other functional ingredients such as penetration enhancers, pH adjusters and stabilizers. These help determine the properties of liposome drugs and influence the quality, pharmacokinetic and pharmacodynamic properties, and safety profile of the liposome drug.

A pre-formulation literature study focuses on the selection of the correct concentration and quality of phospholipids, stabilizers, penetration enhancers and other excipients. Furthermore, a study into the compatibility of the selected auxiliary agents/excipients is planned. In a first phase, binary mixtures are prepared, brought to stability and checked whether degradation is observed. The aim of the study is to extensively investigate the effect of a wide range of excipients on the stability of testosterone, thus identifying which excipients should be avoided in order to have a stable formulation. In addition, the study provides information on whether preservatives and/or antioxidants can/should be added to stabilize the formulation, or whether already stable formulations are obtained in combination with specific excipients.

Based on the results of this stability data, a series of multi-component mixtures with suitable excipients are studied. Safety and compatibility research is ongoing to reduce the risk of surprises in later stages. A better understanding of the excipients and the overall formulation ensures that certain problems and costly rework of formulation steps are avoided during the scale-up to commercialization.

When testing different excipients and combinations of excipients, a visual assessment of physical visual assessment of physical properties is a good initial screening. pH, viscosity, density, assay and homogeneity are assessed.

Different grades of phospholipids, stabilizers (polymers such as Poloxamer and Carbopol), penetration enhancers (alcohols, glycols, etc.) are selected, and work is underway to rationalize the selection to achieve the desired properties in our liposomal formulation.

It is also critical to examine patient expectations regarding the product's characteristics (viscosity, stickiness) and translate these into the intended specifications for development.

### Task 2.2: Formulation Study

Various formulations of the liposomal preparation of the present invention are under investigation, which should have the appropriate viscosity and stability profile.

The formulation study includes the following subtasks:
Determining the intended product specifications, i.e. a stable and well-defined formulation for topical (transdermal) use and with the correct pH, viscosity, testosterone concentration and release profile;
Determining rational qualitative and quantitative formulations suitable for use and production approach: consulting literature for comparative products, selecting excipients, ordering and assessing compatibility;
Carrying out a prototype formulation study on a laboratory scale—batches with different excipients are tested and evaluated.

The formulas with the best properties (viscosity, homogeneity and in vitro release profile) are further subjected to intensive characterization.

Task 2.2 Thus Results in an Innovative Liposomal Testosterone Formulation within the Target Profile.

### Task 2.3: Liposomal Particle Size, Distribution and Zeta Potential

Mean liposome size and distribution are analyzed using dynamic light scattering (DLS) and a Beckman Coulter (Delsa™ NanoS). The sample is diluted with the appropriate solvent if necessary. The size measurements are made at a scattering angle of 90° at 25±2° C.

The zeta potential is measured by Electrophoretic Light Scattering (ELS) (Zetasizer Nano series, Malvern Panalytical, Westborough, MA). All formulation samples are analyzed at room temperature without further treatment. All observations are made in triplicate for the test formulation.

### Task 2.3 Thus Yields the Liposomal Particle Size, Particle Distribution and Zeta Potential.

### Task 2.4: Morphology of the Particles, Loading of the Drug (Testosterone) and Entrapment Efficiency of the Test Formulation

The drug dosage and entrapment efficiency (EE) (also known as encapsulation efficiency) of liposomal formulations are critical parameters that need to be optimized as they affect the dosage, drug release and overall cost of a liposomal drug formulation. The greater the EE, the greater the amount of drug that can be encapsulated and transported to the target tissues.

The drug loading and EE of the liposomes are determined by an ultrafiltration method. The liposomes (0.5 ml) will be placed in an Amicon® Ultra-4 centrifugal filter unit (3000 MWCO, Millipore Centrifugal filters, Millipore, MA) and centrifuged at 5000 rpm for 10 min. The free drug in the filtrate is diluted to approximately 1 ml with an appropriate solvent and analyzed by the validated HPLC methods described in Example 3. The % EE is calculated using the following equation:

$$EE \% = \frac{De - Df}{De} \times 100 \%$$

where De is the total amount of testosterone in the liposomes and Df is the amount of free drug (testosterone) in the filtrate. Another way to express the above equation is:

$$EE ( \% ) = ( amount of drug in liposomes \times 100)/amount of drug used to make liposomes$$

The amount of drug in the liposomes is determined by multiplying the concentration of drug in the filtered liposomes by the total liposome volume.

The drug loading percentage is calculated as shown below:

$$\% drug loading = ( amount of drug in liposomes \times 100)/(amount ( drug + lipids ) used in making the liposomes )$$

All observations are performed in triplicate for the test formulation.

The morphology of liposomes is observed by transmission electron microscopy (JEOL JEM-1230, Japan). Samples diluted with an ethanol solution are applied to copper grids. Excess sample is removed, and the grid is negatively stained with a drop of phosphosulfuric acid (2% w/v). The grid is thoroughly air dried and the samples are analyzed at 25±2° C.

Task 2.4 thus provides the morphology of the particles, loading of the drug (testosterone) and entrapment efficiency of the test formulation

The measured entrapment efficiency is as follows:

| | T (° C.) | Entrapment efficiency (EE) |
|---|---|---|
| Testosterone 1% | 25.0 | 99.8% ± 0.000248 |
| Testosterone 2.5% | 25.0 | 99.9% ± 0.000249 |
| Placebo | 25.0 | / |

A measured EE of more than 99% is an extremely high value, indicating that the liposomes are extremely efficient in incorporating/encapsulating the hormones.

Task 2.5: Investigation of the Release of Testosterone from the Test Formulation

A dialysis bag (3000MWCO, Sigma, Ronkonkoma, NY) is used to test the release of testosterone from the test formulation. The cellulose acetate membrane is pre-activated in doubly distilled water (molecular weight cutoff 3,000 Da) and in a Franz diffusion cell (LOGAN FDC-24, Somerset, NJ) with an effective diffusion area of 0.64 cm². The test formulation (0.2 ml) is added to the donor compartment and 5 ml of freshly prepared buffer (containing solubilizer to improve testosterone solubility) is added to the receptor compartment. The vials are kept at 37±0.5° C. and stirred with a magnetic bar at 300 rpm. The released medium (0.3 ml) is collected at 1, 3, 5, 7, 9, 12, 24, 36 and 48 hours (or longer if necessary) and 0.3 ml of fresh medium is returned to the main medium at each time point. The concentration of testosterone is analyzed by validated HPLC methods as described in Example 1. The amount of drug released from the formulation is calculated using the following equation:

$$Q = ( {C}_{n} \times V + {\sum }_{i = 1}^{n - 1} {C}_{i} \times {V}_{i} ) / A$$

where Cn is the drug concentration in the receptor medium at time n, Ci the drug concentration in the receptor medium, A the effective diffusion area (0.64 cm²), V is the volume of receptor medium (5 ml) and Vi is the sampling volume at time i. All observations are made in triplicate (minimum) for the test formulation.
Task 2.5 Thus Concerns a Study of the Release of Testosterone from the Test Formulation.

### Task 2.6: Rheological Investigation of the Test Formulation of Liposomal Testosterone

The oscillatory rheological measurements are performed on a Kinexus Ultra+ (Malvern Pananalytical Ltd., USA) rotational rheometer with rSpace software (ver. 1.75.2326, Malvern Pananalytical Ltd., USA). The samples are tested using a stainless steel plate/plate geometry (20 mm). The measuring gap is set to 1.0 mm. Prior to the experiments, the samples are placed on the bottom plate. After lowering the top plate, the excess is removed with a spatula. Each measurement is performed in triplicate with fresh samples and the mean values of the parameters are reported.

The measurements, including oscillatory stress sweeping (SS) and temperature sweeping (TS), are performed sequentially in one run. After placing in the rheometer, the samples are equilibrated for 2 minutes at 32.0±0.5° C. Thereafter, the SS study is performed in the range of 1.0-12,000 Pa. In the second step, the samples are cooled to 5.0±0.5° C. and a TS integrator is conducted in the range of 5.0-30.0° C., at a constant stress of 2.0 Pa. For both SS and TS measurements, the oscillation frequency will be set to 1.0 Hz.

The results for SS studies were presented as the dependence of storage (G′) and loss (G″) moduli vs. oscillatory stress (T), plotted in a logarithmic scale. In addition, the G′/G″ cross-over points were calculated. The results of temperature measurements were plotted as G′ dependence on temperature.

### Statistical Analysis of the Data

The data is reported as means t SD. The results obtained were analyzed by one-way analysis of variance (ANOVA) followed by post-hoc Tukey test. The statistical significance level in all tests was set at 5%. All calculations were performed with JMP® Pro 14.2.0 (SAS Institute Inc., Cary, NC, USA).

Task 2.6 thus concerns rheological analyses of the pilot formulation of the liposomal preparation with encapsulated testosterone of the invention.

### Example 5: Stability Study on the Liposomal Preparation of the Present Invention

The main objective of the present experiment is to further characterize the pilot liposomal testosterone formulation for its stability. This goal is achieved by subjecting the test formulation to two conditions: 25° C./60% relative humidity (RH) and 40° C./75% RH for a period of 90 days (on one batch of the test formulation). For each condition, the following properties are assessed and recorded over the 90 days at the following 30-, 60- and 90-day time points: a) liposomal size and distribution (Task 3.1) and b) testosterone content in the formulation (Task 3.2). The drug content is determined by the validated HPLC method as described in Example 3. Data is collected to gain a thorough understanding of the stability under the various conditions leading up to the development of the final product.

### Task 3.1: Liposomal Particle Size and Distribution

The average size of the liposomes is analyzed using dynamic light scattering (DLS) and a Beckman Coulter (Delsa™ NanoS). The sample is diluted if necessary with the appropriate solvent. The size measurements are performed at a scattering angle of 90° at 25° C.±2. All formulation samples are analyzed at room temperature without further Treatment. All Observations are Made in Triplicate for the Test Formulation.

Task 3.2: Testosterone content of liposomes in the test formulation

The amount of drug in the vesicles at each time point is determined using the approach and methods already described in Example 4 Task 2.2. All observations are performed in triplicate for the test formulation. The testosterone is analyzed by HPLC using methods described in Example 3.

### Statistical Analysis of the Data

The data is reported as means t SD. The results obtained were analyzed by one-way analysis of variance (ANOVA) followed by post-hoc Tukey test. The statistical significance level in all tests was set at 5%. All calculations were performed with JMP® Pro 14.2.0 (SAS Institute Inc., Cary, NC, USA).

To analyze the stability of the test formula during storage, a stability study is performed at 25° C./60 RH and 40° C./75 RH over a period of 90 days. The testosterone content in the vesicles and the size distribution are evaluated after 30, 60 and 90 days.

In addition, an in-use study is conducted. The aim of this study is to assess the risk of microbiological contamination, proliferation and/or physico-chemical degradation after repeated opening of the vials containing the liposomal preparation. The vials are opened in the morning each day and 1 gram of product is taken from the vials to simulate daily use for 1 week. In between, the vials are stored in both conditions and tested at each time point. Data is collected to provide a thorough understanding of stability under the various conditions leading up to the development of the final product.

### Example 6: Optimization of the Liposomal Formulation and Production Method (QbD)

The goal of the present example is to arrive at a meaningful product with the right quality specifications to achieve a significantly higher and long-lasting bioavailability with a superior steady state of total testosterone.

QbD (Quality by Design) is a systematic approach used to improve product quality, better understand the process and support adequate regulatory compliance for vehicle-based drug delivery systems commonly used in the pharmaceutical industry. The goal of this research is to optimize critical material attributes and critical process parameters to effectively develop a liposomal drug delivery system for use in testosterone transdermal application.

The variables considered are the critical material attributes (concentration and types of excipients) and critical process parameters (CPPs) (mixing time, mixing speed and addition rate) variables.

The Experimental Design (Design of Experiments—DoE) is a logical approach to multivariate statistics that aims to guide scientific research to study the effects of several factors simultaneously, not only individually but also on their interactions. A full-factorial experimental study design allows researchers to systematically study the response of different factors, the relationships between factors and the interactions between factors by effectively quantifying the scientific error. To select the best fit during computer modeling, a statistical tool is used to gain a detailed understanding of the results obtained using a QbD approach.

Over the years, the pharmaceutical industry has made significant efforts to ensure product quality, comply with regulations and produce pharmaceutical products as cost-effectively as possible. Therefore, advanced processes and technologies are applied that require a balance between operational complexity and scientific progress. QbD is a new advanced systematic risk-based approach applied to pharmaceuticals to develop formulations with predefined objectives and enhance product and process understanding with improved safety and stability. Application of the QbD approach is considered to have a significant regulatory impact on drug quality as opposed to older empirical processes which carry a higher risk of failure or sub-optimal quality. The US Food and Drug Administration (FDA) requires pharmaceutical companies to apply risk-based approaches and QbD principles in product development, from research and development (R&D) to the production cycle. This makes it a desirable process to avoid product failure during production and to meet regulatory requirements.

### Task 4.1: Defining the Quality Target Profile (QTPP) and Critical Quality Attributes (CQAs)

According to the principles of QbD, the methodology starts with establishing the Quality Target Product Profile (QTPP) and identifying the Critical Quality Attributes (CQAs) needed to meet it.

### Task 4.2: Analytical Development

Analytical methods required to test the specifications and the physical and chemical properties of the formulation after production are developed and validated.

These methods include methods for determining testosterone levels and testosterone impurities (testosterone impurities assay). In addition, methods are being developed for the determination of preservatives, for the determination of viscosity, pH and density.

### Task 4.3: Evaluation of CMAs and CPPs.

The critical process parameters (CPPs) and critical material attributes (CMAs) are evaluated to determine their influence on the CQAs. DoE is used for risk evaluation and optimization and to understand the effects of various variables on the response. Experimental trials will be defined using full factorial designs to achieve the optimized formulation. The process is finally validated and tested for robustness.

### Task 4.4: Microbiological Safety

Since the presence of certain micro-organisms in non-sterile preparations has the potential to reduce or inactivate the therapeutic effect of the product and to adversely affect or harm the patient's health, a low biological burden should be ensured of the finished dosage/administration form by applying current Good Manufacturing Practice (GMP) guidelines during the manufacture, storage and distribution of the finished product. In addition, a method for microbial investigation of the formula will be developed, performed according to the guidelines and validated.

### Task 4.5: Preservative Efficacy Test (PET)

A microbial test (PET) is performed on the formulation of the liposomal preparation to ensure that the preservative retains its ability to inhibit microorganisms and to determine whether the addition of a preservative is necessary in the commercial product.

### Example 7: Tolerability Study of the Liposomal Preparation of the Present Invention in Transdermal Application

The main objective is to investigate the cytotoxicity and irritation of the optimized liposomal preparation with encapsulated testosterone. Human keratinocytes and fibroblasts are used for the initial cytotoxicity tests in Task 5.1. Finally, a full-thickness human bioengineered skin model is used to test the irritating potential of the test formulation (Task 5.2). The overall goal is to demonstrate that the formulation is safe for topical skin applications.

### Task 5.1: Cytotoxicity of Test Formulation Using a Human Keratinocyte Cell Line (HaCaTs) and Human Fibroblasts

HaCaT (a human keratinocyte cell line) is cultured in Dulbecco's Modified Eagle Medium (DMEM) supplemented with 10% fetal bovine serum (FBS) and 1% penicillin/streptomycin (37° C., 10% CO₂, RH 95%). When 90% confluency is reached, the cells are rinsed with distilled phosphate buffered saline (PBS), trypsinized, and passaged at a 1:4 ratio. HaCaTs with a passage number lower than 50 will be used in the study. 100 μl HaCaT cell suspension is added to each well in a 96-well cell culture plate; the seeding density will be approximately 2000 cells per well (cell number measured with a Cellometer Auto A4, Nexcelom Bioscience, MA). The cells are incubated for 4 hours to ensure adequate attachment, after which the media in all wells is changed to the experimental conditions described below:
(a) Test formulation (with drug and liposome) (in triplicate); in addition, as a control, three wells are prepared with cell culture medium containing liposome-free and drug-free formulation/hydrogel, 100% viability controls.
(b) Testosterone dissolved in the culture medium using a solvent such as 0.1% v/v DMSO (in hexaplicates). Cell culture medium containing 0.1% v/v DMSO is used as 100% viability.
(c) Cell culture medium, cell culture medium with 10% DMSO, and 10% AlamarBlue® containing medium are used as negative control, positive control, and blank, respectively (in hexaplicates).

Cells subjected to these experimental conditions are cultured for 3 days, after which cell viability is measured using the AlamarBlue® metabolic assay. Cell culture medium with 10% AlamarBlue® reagent is added and fluorescence intensity is measured (560 nm/590 nm; manual amplification 97%) when the negative controls begin to turn pink.

The cytotoxicity data and the half-maximal inhibitory concentration (IC50) are determined according to the protocol provided with the AlamarBlue® assay reagent (AbD Serotec).

The above protocol (adapted if necessary) will be applied for a second study, but this time human fibroblasts will be used instead of the HaCaTs.

### Task 5.2: In Vitro Skin Irritation Test Using a Three-Dimensional EpiDermFT Skin Model MTT Assay on the 3D EpiDermFT Skin Model:

The in vitro irritation study will be conducted according to the protocol of the manufacturer MatTek Corporation, Ashland, MA, USA. The EpidermFT skin model is a three-dimensional human skin model with intact skin barrier function and is made up of human skin cells (fibroblasts and keratinocytes) and includes all layers of the skin: stratum corneum, epidermis and dermis. The model has no appendages (hair follicles and sweat glands) or microcapillary blood circulation. The EpiDermFT reconstructed human skin barrier is placed overnight at 37° C. and 5% CO₂ in a humidified incubator. Next, the test formulation with drug (testosterone) is encapsulated in liposomes and testosterone alone included in the hydrogel base, gel base, blank liposomes in the gel base, 5% (w/v) sodium dodecyl sulfate (SDS), and Dulbecco's Phosphate Buffered Saline (DPBS) are used as the formulation test sample, positive, and negative controls, respectively. All test formulations are directly applied to the top of the 3D tissue, which is exposed for 60 minutes. Subsequently, the EpiDermFT is rinsed with distilled PBS, then transferred to fresh medium and incubated for 42 hrs. All tests are performed in triplicate.

At the end of the incubation (42 hours), the skin tissues are transferred to a 24-well plate pre-filled with 300 μL MTT medium (1 mg/ml) per well and placed for 3 hours in a 37° C., 5% CO₂-humidified incubator. The skin tissues are then removed from the MTT medium. The formazan salt, which results from the MTT dye and the viable cell reaction, is extracted from the tissues by transferring them to a 24-well plate containing 2 ml of isopropanol. The immersed tissues are shaken at 120 rpm at room temperature for 2 hours. After the extraction period, the optical density (OD) of the extracted formazan is determined by transferring 200 μl of each extraction solution to a new optically clear 96-well plate. Isopropanol is used as blank. The OD values are determined with a spectrophotometer at 570 nm. The percent viability of the cells is calculated using the following equation:

$$\% viability = [ OD ( sample ) \times 100 ] / OD ( negative control )$$

### Statistical Analysis of the Data

The data are reported as mean±SD. The results obtained will be analyzed with a one-way analysis of variance (ANOVA) followed by a post-hoc Tukey test. The statistical significance level in all tests will be set at 5%. All calculations will be performed with JMP® Pro 14.2.0 (SAS Institute Inc., Cary, NC, USA).

### Example 8: Pharmacokinetic Study of Three Formulations of the Preparation According to the Present Invention with Different Concentrations of Encapsulated Testosterone

The purpose of this is to select the formulation with the best testosterone concentration, which best meets the required criteria (including irritation profile), and which is suitable for use in further human studies. This formulation also outperforms current marketed products.

### Task 6.1: Design Synopsis of the Planned Study

Review of existing information on other similar products under development, including regulatory and market, existing clinical trial data, labeling, etc., to prepare a design synopsis of the planned study.

### Task 6.2: Study on 5 Healthy Subjects.

For this study, the subjects must first undergo treatment with an LHRH (luteinizing hormone-releasing hormone) agonist plus an aromatase inhibitor to completely switch off their endogenous testosterone production. This is the best way to prepare for the PK study.

Testosterone blood level determination is performed by LC:MS.

The present invention should not be construed as being limited to the embodiments described above and certain modifications or changes may be added to the examples described without having to re-evaluate the appended claims. For example, the present invention has been described with reference to testosterone, but it should be understood that the invention may be applied (subject to modifications to the testing protocol) to any other hormone, such as but not limited to any of the sex hormones described in the above embodiments.

1. A method for producing a liposomal preparation by emulsification, the preparation containing liposomes with encapsulated hormones or ester thereof, comprising:
a) a first emulsification step of emulsifying a first composition, the first composition comprising water, propylene glycol, an emulsifier and a hormone to be encapsulated; and optionally
b) a second emulsification step wherein the emulsified composition of step a) is added to a second composition and emulsified, the second composition comprising glycerin, water and a polymer, characterized in that for the emulsification of the first emulsification step, and optionally for the emulsification of the second emulsification step, a rotor-stator emulsifier is used, which is provided with a stator with perforations with a diameter between 0.8 and 5 mm, and wherein the rotational speed is at least 6000 rpm.

2. The method according to claim 1, wherein the hormones are sex hormones or esters thereof, wherein the sex hormones are selected from the group of testosterone, hydrocortisone, estradiol and progesterone and esters thereof.

3. The method according to claim 1, wherein the perforations of the stator have a diameter comprised between 0.8 and 3.0 mm, and optionally wherein a smallest distance from a first to a second perforation is 0.5 to 1.5 mm.

4. The method according to claim 1, wherein 85% of the liposomes of the liposomal preparation have a diameter or particle size comprised between 160 and 180 nm.

5. The method according to claim 1, wherein the emulsifier is a phosphatidylcholine from lecithin.

6. The method according to claim 1, wherein the polymer is a neutralized carbomer.

7. The method according to claim 1, wherein the first composition is emulsified for at least 15-25 minutes.

8. The method according to claim 1, wherein the method includes the second composition, and wherein the emulsified composition of step a) is emulsified together with the second composition for at least 10 to 20 minutes.

9. The method according to claim 1, wherein the polypropylene glycol and emulsifier are present in a 1:1 ratio, and optionally in the case of a second emulsification step where the polypropylene glycol, emulsifier and glycerin are present in a 1:1:1 ratio.

10. A liposomal preparation comprising liposomes with encapsulated hormones or esters thereof, wherein the composition comprises water, propylene glycol, an emulsifier, a hormone to be encapsulated, glycerin, and a polymer such as a neutralized carbomer, wherein the liposomes have a single bilayer, and wherein at least 85% of the liposomes have a diameter or particle size which is comprised between 160 and 180 nm.

11. The liposomal preparation according to claim 10, wherein the polypropylene glycol and emulsifier are present in a 1:1 ratio, optionally wherein polypropylene glycol, emulsifier and glycerin are present in a 1:1:1 ratio.

12. The liposomal preparation according to claim 10, wherein the hormones are sex hormones or esters thereof, wherein the sex hormones are selected from the group of testosterone, hydrocortisone, estradiol and progesterone and esters thereof.

13. The liposomal preparation obtained by the method according to claim 1.

14. Using the liposomal preparation according to claim 10, for use in hormone therapy or for use in the treatment of a patient requiring hormone therapy.

15. Using the liposomal preparation according to claim 10, for use in the treatment of hypogonadism and/or adrenal insufficiency.

16. Using the liposomal preparation according to claim 14, wherein the liposomal preparation is administered per os (PO), via intravenous administration (IV) or via transdermal administration, optionally wherein the administration is repeated one or more times.

17. Using the liposomal preparation according to claim 15, wherein the liposomal preparation is administered per os (PO), via intravenous administration (IV) or via transdermal administration, optionally wherein the administration is repeated one or more times.
